(19) United States
(12) Patent Application Publication
(10) Pub. No.: US 2007/0209660 A1
(43) Pub. Date: Sep. 13, 2007

(54) Intranasal Opioid Compositions, Delivery Devices and Methods of Using Same

(76) Inventor: Daniel P. Wermeling, Lexington, KY (US)

Correspondence Address: GOODWIN PROCTER LLP;PATENT ADMINISTRATOR, EXCHANGE PLACE, BOSTON, MA, 02109-2881, US

(21) Appl. No.: 11/674,787

(22) Filed: Feb. 14, 2007

Related U.S. Application Data
(63) Continuation of application No. 11/243,613, filed on Oct. 5, 2005.
Application No. 11/243,613 is a continuation-in-part of application No. 10/647,789, filed on Aug. 25, 2003.
Application No. 10/647,789 is a continuation-in-part of application No. 09/790,199, filed on Feb. 20, 2001, now Pat. No. 6,610,271.
Application No. 09/790,199 is a continuation-in-part of application No. 09/569,125, filed on May 10, 2000, now abandoned.

(51) Int. Cl. A61M 11/00; A61K 31/00

(52) U.S. Cl. 128/200.14; 514/282

(57) ABSTRACT

The present invention relates to pharmaceutical compositions comprising opioids and a liquid nasal carrier, to delivery devices comprising such compositions, and to methods of manufacture and use of such compositions.

This application is a continuation-in-part of U.S. application Ser. No. 10/647,789, which is a continuation-in-part of U.S. application Ser. No. 09/790,199 filed Feb. 20, 2001 (now U.S. Pat. No 6,620,372), which is a continuation-in-part of U.S. application Ser. No. 09/569,125 filed May 10, 2000, now abandoned. The entire disclosure of these applications are hereby individually incorporated by reference herein in their entirety.

### BACKGROUND OF THE INVENTION

Pain is a major symptom of many diseases including, for example, cancer, arthritis, neurological diseases, heart attacks, etc. Inadequate treatment of pain can lead to depression, anger, fear of disease progression and in some extreme cases, suicide.

Non-compliance is a particular problem in pain medication since pain treatment regimens often involve administering medications by injection (e.g., intravenous (IV), intramuscular (IM) or subcutaneous injection). The intravenous route, in particular, is regarded as one of the most inconvenient routes to administer pain medication to achieve rapid pain relief. Intravenous administration can also cause non-compliance due to fear of injection and to unpleasant injection site side effects such as pain, irritation and infection.

Among the many medications available to treat pain, opioids (e.g., morphine, methadone, hydromorphone, butorphanol, etc.) play an important role. Opioids have an extensive history of use and are generally more effective in treating severe pain than other medications such as aspirin, acetaminophen, ibuprofen, etc. Further, opioids exhibit few adverse effects on organs such as the stomach, liver, or kidney, other than very minor problems such as nausea or constipation which contrasts with other pain medications such as aspirin or anti-inflammatory drugs that may cause ulcers, kidney problems, high blood pressure, or liver inflammation. In addition to relieving pain, opioids have other beneficial effects such as peripheral arterial vasodilation that can provide the benefit of reducing oxygen demand on the heart in treatment of heart attacks.

Given the problems associated with inadequate treatment of pain and patient non-compliance, there is a need for opioid compositions that address one or more of the above described drawbacks associated with injectable dosage forms.

### SUMMARY OF THE INVENTION

In one embodiment, the present invention provides a pharmaceutical composition for intranasal administration to a mammal comprising a therapeutically effective amount of an opioid, a liquid nasal carrier for the opioid, and optionally one or more pharmaceutically acceptable excipients.

The related terms “therapeutically effective amount,” “prophylactically effective amount,” or “effective amount” as used herein refer to an amount of drug or agent that is sufficient to elicit the required or desired therapeutic and/or prophylactic response, as the particular treatment context may require.

In another embodiment, the present invention provides a method of treating a mammal suffering from pain comprising intranasally administering to the mammal an effective amount of a composition as described herein.

In another embodiment, the present invention provides an intranasal unit-dose delivery device comprising one or more sealed vessels or containers containing a sterilized, preservative-free pharmaceutical composition. The composition comprises an effective amount of butorphanol tartrate (or other opioid) and a liquid nasal carrier. In a related embodiment, upon positioning the device a fixed distance away from a detection laser beam, actuating the device to produce a spray plume perpendicular to the laser beam, and detecting droplet size distribution of the spray plume with the laser beam, the spray plume has defined droplet size dispersion characteristics.

In another embodiment, upon positioning the device a fixed distance away from an impaction plate, actuating the device to produce a spray pattern onto the impaction plate, and measuring the diameter of the spray pattern, the spray pattern has a defined maximum diameter, minimum diameter and/or span.

### BRIEF DESCRIPTION OF THE DRAWINGS

FIG. 1 is a graphic representation of the concentration of butorphanol in blood plasma versus time for a butorphanol composition as administered using a unit-dose delivery device or a multi-dose delivery device.

FIG. 2 is a graphic representation of the data of FIG. 1 over a longer time period.

FIG. 3 is a graphic representation of the concentration of hydromorphone in blood plasma versus time for IV, IM and intranasal (IN) doses.

FIG. 4 is a graphic representation of the data of FIG. 3 over a longer period of time.

FIG. 5 is a graphic representation of the concentration of hydromorphone in blood plasma versus time for a group of subjects.

### DETAILED DESCRIPTION OF THE INVENTION

While the present invention is capable of being embodied in various forms, the description below of several embodiments is made with the understanding that the present disclosure is to be considered as an exemplification of the invention, and is not intended to limit the invention to the specific embodiments illustrated. Headings are provided for convenience only and are not to be construed to limit the invention in any way. Embodiments illustrated under any heading may be combined with embodiments illustrated under any other heading.

The use of numerical values in the various ranges specified in this application, unless expressly indicated otherwise, are stated as approximations as though the minimum and maximum values within the stated ranges were both preceded by the word “about.” In this manner, slight variations above and below the stated ranges can be used to achieve substantially the same results as values within the ranges. As used herein, the terms “about” and “approximately” when referring to a numerical value shall have their plain and ordinary meanings to one skilled in the art of pharmaceutical sciences or the art relevant to the range or element at issue. The amount of broadening from the strict numerical boundary depends upon many factors. For example, some of the factors to be considered may include the criticality of the element and/or the effect a given amount of variation will have on the performance of the claimed subject matter, as well as other considerations known to those of skill in the art. Thus, as a general matter, “about” or “approximately” broaden the numerical value. For example, in some cases, “about” or “approximately” may mean ±5%, or ±10%, or ±20%, or ±30% depending on the relevant technology. Also, the disclosure of ranges is intended as a continuous range including every value between the minimum and maximum values.

Opioids

In various embodiments, compositions of the invention comprise an opioid. The term “opioid” as used herein includes any substance naturally or synthetically derived from opium. Suitable opioids for use in the present invention include, but are not limited to, morphine, apomorphine, dihydromorphine, diacetylmorphine, hydromorphone, hydrocodone, oxymorphone, levorphanol, levallorphan, levophenacylmorphan, norlevorphanol, nalorphine, nalbuphine, buprenorphine, butorphanol, naloxone, methadone, hydrocodone, oxycodone, diacetylmorphine, naltrexone, nalmexone, oxycodone, oxilorphan, cyclorphan, ketobemidone, fentanyl, sufentanil, alfentanyl, or combinations thereof.

The opioid may be in free form or in pharmaceutically acceptable salt or complex form. Non-limiting examples of pharmaceutically acceptable salts of opioids include those salt-forming acids and bases that do not substantially increase the toxicity of the compound. Non-limiting examples of suitable salts include salts of alkali metals such as magnesium, potassium and ammonium, salts of mineral acids such as hydrochloric, hydriodic, hydrobromic, phosphoric, metaphosphoric, nitric and sulfuric acids, as well as salts of organic acids such as tartaric, acetic, citric, malic, benzoic, glycollic, gluconic, gulonic, succinic, arylsulfonic, e.g. p-toluenesulfonic acids, and the like.

Compositions of the invention can comprise one or more opioids in any suitable amount. In one embodiment, a composition of the invention comprises an opioid in an amount of about 1 μg to about 100 mg, about 1 μg to about 80 mg, about 1 μg to about 50 mg or about 1 μg to about 40 mg. Compositions of the invention typically comprise one or more opioids in a concentration of about 0. 1 mg/ml to about 300 mg/ml, about 0.5 mg/ml to about 250 mg/ml, about 0.75 mg/ml to about 200 mg/ml, or about 1 mg/ml to about 100 mg/ml.

Generally speaking, the maximal dosage of a pharmaceutical composition of the present invention for a mammal is the highest dosage that elicits analgesia or anesthesia, yet which does not cause undesirable or intolerable side effects such as respiratory depression. The minimal dose of such a composition is generally the lowest dose that achieves the desired result, for example suitable analgesia or anesthesia. One of ordinary skill in the art will readily appreciate the doses of various opioids that are effective to achieve the pain relieving effect in the mammal. Typical doses of opioids for intranasal administration include, but are not limited to, hydromorphone HCl from about 0.1 mg to about 30 mg or about 1 mg to about 15 mg; butorphanol tartrate from about 0.1 mg to about 10 mg or about 1 mg to about 5 mg; fentanyl citrate from about 5 μg to about 500 μg or about 10 μg to about 250 μg; methadone HCl from about 0.5 mg to about 50 mg or about 1 mg to about 30 mg; oxymorphone HCl from about 0.1 mg to about 30 mg or about 1 mg to about 20 mg; and morphine HCl from about 1 mg to about 40 mg or about 5 mg to about 30 mg. In one embodiment, compositions of the invention comprise one or more of the foregoing amounts of opioid.

Liquid Nasal Carrier

Compositions of the present invention comprise a liquid nasal carrier. As used herein, the phrase “liquid nasal carrier” refers to a liquid vehicle (e.g. solution, emulsion, or suspension) designed for delivery of an opioid to the nasal mucosa of a subject. The liquid nasal carrier can include one or more diluents suitable for application to the nasal mucosa. Suitable diluents include aqueous or non-aqueous diluents or combination thereof. Examples of aqueous diluents include, but are not limited to, saline, water, water for injection (WFI), dextrose or combinations thereof. Illustrative non-aqueous diluents include, but are not limited to, alcohols, particularly polyhydroxy alcohols such as propylene glycol, polyethylene glycol, glycerol, and vegetable and mineral oils. These aqueous and/or non-aqueous diluents can be added in various concentrations and combinations to form solutions, suspensions, oil-in-water emulsions or water-in-oil emulsions. The liquid nasal carrier can be present in any suitable amount, for example about 10% to about 99%, about 20% to about 98%, about 30% to about 97%, by weight. In another embodiment, the liquid nasal carrier can be added to the other components of the composition in an amount sufficient to q.s. the formulation to a desired volume.

Pharmaceutical Excipients

Compositions of the invention optionally comprise one or more pharmaceutically acceptable excipients. The term “excipient” herein means any substance, not itself a therapeutic agent, used as a carrier or vehicle for delivery of a therapeutic agent to a subject or added to a pharmaceutical composition to improve its handling or storage properties or to permit or facilitate formation of a unit dose of the composition.

Illustrative excipients include antioxidants, surfactants, co-solvents, adhesives, agents to adjust the pH and osmolarity, preservatives, antioxidants, thickening agents, sweetening agents, flavoring agents, taste masking agents, colorants, buffering agents, and penetration enhancers. Generally speaking, a given excipient, if present, will be present in an amount of about 0.001% to about 20%, about 0.01% to about 10%, about 0.02% to about 5%, or about 0.3% to about 2.5%, by weight.

Illustrative antioxidants for use in the present invention include, but are not limited to, butylated hydroxytoluene, butylated hydroxyanisole, potassium metabisulfite, and the like. One or more antioxidants, if desired, are typically present in a composition of the invention in an amount of about 0.01% to about 2.5%, by weight.

In various embodiments, compositions of the invention comprise a preservative. Ideally, the optional preservative will be present in quantities sufficient to preserve the composition, but in quantities low enough that they do not cause irritation of the nasal mucosa. Suitable preservatives include, but are not limited to, benzalkonium chloride, methyl, ethyl, propyl or butylparaben, benzyl alcohol, phenylethyl alcohol, benzethonium, or combination thereof. Typically, the optional preservative is present in an amount of about 0.01% to about 0.5% or about 0.01% to about 2.5%, by weight.

In other embodiments, compositions of the invention are preservative-free. As used herein, the term “preservative-free” includes compositions that do not contain any preservative. Thus, the composition does not contain, for example, benzalkonium chloride, methyl, ethyl, propyl or butylparaben, benzyl alcohol, phenylethyl alcohol, or benzethonium.

In one embodiment, compositions of the invention optionally comprise a buffering agent. The optional buffering agent, if present, is present in a composition of the invention in an amount that does not irritate the nasal mucosa. Buffering agents include agents that reduce pH changes. Illustrative classes of buffering agents for use in various embodiments of the present invention comprise a salt of a Group IA metal including, for example, a bicarbonate salt of a Group IA metal, a carbonate salt of a Group IA metal, an alkaline earth metal buffering agent, an aluminum buffering agent, a calcium buffering agent, a sodium buffering agent, or a magnesium buffering agent. Other suitable classes of buffering agents include alkali (sodium and potassium) or alkaline earth (calcium and magnesium) carbonates, phosphates, bicarbonates, citrates, borates, acetates, phthalates, tartrates, succinates and the like, such as sodium or potassium phosphate, citrate, borate, acetate, bicarbonate and carbonate.

Non-limiting examples of suitable buffering agents include aluminum, magnesium hydroxide, aluminum glycinate, calcium acetate, calcium bicarbonate, calcium borate, calcium carbonate, calcium citrate, calcium gluconate, calcium glycerophosphate, calcium hydroxide, calcium lactate, calcium phthalate, calcium phosphate, calcium succinate, calcium tartrate, dibasic sodium phosphate, dipotassium hydrogen phosphate, dipotassium phosphate, disodium hydrogen phosphate, disodium succinate, dry aluminum hydroxide gel, magnesium acetate, magnesium aluminate, magnesium borate, magnesium bicarbonate, magnesium carbonate, magnesium citrate, magnesium gluconate, magnesium hydroxide, magnesium lactate, magnesium metasilicate aluminate, magnesium oxide, magnesium phthalate, magnesium phosphate, magnesium silicate, magnesium succinate, magnesium tartrate, potassium acetate, potassium carbonate, potassium bicarbonate, potassium borate, potassium citrate, potassium metaphosphate, potassium phthalate, potassium phosphate, potassium polyphosphate, potassium pyrophosphate, potassium succinate, potassium tartrate, sodium acetate, sodium bicarbonate, sodium borate, sodium carbonate, sodium citrate, sodium gluconate, sodium hydrogen phosphate, sodium hydroxide, sodium lactate, sodium phthalate, sodium phosphate, sodium polyphosphate, sodium pyrophosphate, sodium sesquicarbonate, sodium succinate, sodium tartrate, sodium tripolyphosphate, synthetic hydrotalcite, tetrapotassium pyrophosphate, tetrasodium pyrophosphate, tripotassium phosphate, trisodium phosphate, and trometamol. (Based in part upon the list provided in The Merck Index, Merck & Co. Rahway, N.J. (2001)). Furthermore, combinations or mixtures of any two or more of the above mentioned buffering agents can be used in the pharmaceutical compositions described herein. One or more buffering agents, if desired, are present in compositions of the invention in an amount of about 0.01% to about 5% or about 0.01% to about 3%, by weight.

In one embodiment, compositions of the invention optionally comprise one or more surfactants. Optional surfactants are typically present in a composition of the invention in an amount of about 0.1 mg/ml to about 10 mg/ml, about 0.5 mg/ml to 5 mg/ml or about 1 mg/ml.

In various embodiments, compositions the invention may include one or more agents that increase viscosity. Illustrative agents that increase viscosity include, but are not limited to, methylcellulose, carboxymethylcellulose sodium, ethylcellulose, carrageenan, carbopol, and/or combinations thereof. Typically, one or more viscosity increasing agents, if desired, are present in compositions of the invention in an amount of about 0.1% to about 10%, or about 0.1% to about 5%, by weight.

In various embodiments, compositions of the invention comprise one or more sweeteners and/or flavoring agents. Suitable sweeteners and/or flavoring agents include any agent that sweetens or provides flavor to a pharmaceutical composition. The sweetener or flavoring agent will mask any bitter or bad taste that may occur if the pharmaceutical composition drips back into the mouth after intranasal administration. By addition of a sweetener or flavoring agent to the intranasal composition, any barrier that a patient may have to taking the intranasal composition because of unpleasant taste is reduced. Optional sweetening agents and/or flavoring agents are typically present in a composition of the invention in an amount of about 0.1 mg/ml to about 10 mg/ml, about 0.5 mg/ml to 5 mg/ml or about 1 mg/ml.

Illustrative sweeteners or flavoring agents include, without limitation, acacia syrup, anethole, anise oil, aromatic elixir, benzaldehyde, benzaldehyde elixir, cyclodextrins, compound, caraway, caraway oil, cardamom oil, cardamom seed, cardamom spirit, compound, cardamom tincture, compound, cherry juice, cherry syrup, cinnamon, cinnamon oil, cinnamon water, citric acid, citric acid syrup, clove oil, cocoa, cocoa syrup, coriander oil, dextrose, eriodictyon, eriodictyon fluidextract, eriodictyon syrup, aromatic, ethylacetate, ethyl vanillin, fennel oil, ginger, ginger fluidextract, ginger oleoresin, dextrose, glucose, sugar, maltodextrin, glycerin, glycyrrhiza, glycyrrhiza elixir, glycyrrhiza extract, glycyrrhiza extract pure, glycyrrhiza fluidextract, glycyrrhiza syrup, honey, iso-alcoholic elixir, lavender oil, lemon oil, lemon tincture, mannitol, methyl salicylate, nutmeg oil, orange bitter, elixir, orange bitter, oil, orange flower oil, orange flower water, orange oil, orange peel, bitter, orange peel sweet, tincture, orange spirit, compound, orange syrup, peppermint, peppermint oil, peppermint spirit, peppermint water, phenylethyl alcohol, raspberry juice, raspberry syrup, rosemary oil, rose oil, rose water, stronger, saccharin, saccharin calcium, saccharin sodium, sarsaparilla syrup, sarsaparilla compound, sorbitol solution, spearmint, spearmint oil, sucrose, sucralose, syrup, thyme oil, tolu balsam, tolu balsam syrup, vanilla, vanilla tincture, vanillin, wild cherry syrup, or combinations thereof.

Illustrative taste masking agents includes, but are not limited to, cyclodextrins, cyclodextrins emulsions, cyclodextrins particles, cyclodextrins complexes, or combinations thereof.

The foregoing excipients can have multiple roles as is known in the art. For example, some flavoring agents can serve as sweeteners as well as flavoring agent. The classification of excipients above is not to be construed as limiting in any manner.

Pharmaceutical compositions as disclosed herein are not limited to any particular pH. In one embodiment, pH of a composition of the invention ranges from about 3 to about 7, about 3 to about 6, or about 4 to about 6, for example about 5. If adjustment of pH is needed, it can be achieved by the addition of an appropriate acid, such as hydrochloric acid, or base, such as for example, sodium hydroxide.

Pharmaceutical compositions of the invention can be prepared in any suitable manner. In some embodiments, the compositions are prepared by mixing an opioid with a liquid nasal carrier and one or more optional excipients at room temperature under aseptic conditions. In other embodiments, the mixture can be prepared under non-aseptic conditions and then sterile filtered, autoclaved or otherwise sterilized and packaged in a delivery device. It will be understood by those of ordinary skill in the art that the order of mixing is not critical, and the present invention includes without limitation mixing of compositions of the invention in any order.

Pharmacokinetic Profile

In one embodiment, where the drug being delivered is butorphanol, upon intranasal administration of a composition of the invention to a subject, the subject exhibits one or more of: a Tₘₐₓ butorphanol plasma concentration of at latest about 0.75 hr; a Cₘₐₓ butorphanol plasma concentration of at least about 1000 pg/ml, for example about 1000 pg/ml to about 10000 pg/ml; and/or an AUC butorphanol plasma concentration of at least about 3000 pg*hr/ml, for example about 3000 to about 18000 pg*hr/ml. In a related embodiment, the above PK parameters result after administration of a composition in an amount sufficient to provide the subject with about 0.5 to about 1.4 mg of butorphanol moiety.

In a related embodiment, where the drug being delivered is butorphanol, upon intranasal administration of a butorphanol composition of the invention to a subject, the subject exhibits one or more of: a Tₘₐₓ butorphanol plasma concentration of at latest about 0.60 hr; a Cₘₐₓ butorphanol plasma concentration of at least about 1500 pg/ml, for example about 1500 pg/ml to about 9000 pg/ml; and/or an AUC butorphanol plasma concentration of at least about 5200 pg*hr/ml, for example about 6000 to about 17000 pg*hr/ml. In a related embodiment, the above PK parameters result after administration of a composition in an amount sufficient to provide the subject with about 0.5 to about 1.4 mg of butorphanol moiety.

In another embodiment, where the drug being delivered is butorphanol, upon intranasal administration of a butorphanol composition of the invention to a subject, the subject exhibits one or more of: a Tₘₐₓ butorphanol plasma concentration of at latest about 0.50 hr; a Cₘₐₓ butorphanol plasma concentration of at least about 1600 pg/ml, for example about 1600 pg/ml to about 8000 pg/ml; and/or an AUC butorphanol plasma concentration of about 6000 pg*hr/ml, for example about 7000 to about 16000 pg*hr/ml. In a related embodiment, the above PK parameters result after administration of a composition in an amount sufficient to provide the subject with about 0.5 to about 1.4 mg of butorphanol moiety.

In another embodiment, where the drug being delivered is butorphanol, upon intranasal administration of a butorphanol composition of the invention to a subject, the subject exhibits one or more of: a Tₘₐₓ of butorphanol plasma concentration of at latest about 0.40 hr; a Cₘₐₓ butorphanol plasma concentration of at least about 1700 pg/ml, for example about 1700 pg/ml to about 7000 pg/ml; and/or an AUC butorphanol plasma concentration of at least about 7000 pg*hr/ml, for example about 7000 pg*hr/ml to about 15000 pg*hr/ml. In a related embodiment, the above PK parameters result after administration of a composition in an amount sufficient to provide the subject with about 0.5 to about 1.4 mg of butorphanol moiety.

In another embodiment, where the drug being delivered is butorphanol, upon intranasal administration of a butorphanol composition of the invention to a subject, the subject exhibits one or more of: a Tₘₐₓ butorphanol plasma concentration of at latest about 0.30 hr; a Cₘₐₓ butorphanol plasma concentration of at least about 1800 pg/ml, for example about 1800 pg/ml to about 6000 pg/ml; and/or an AUC butorphanol plasma concentration of at least about 8000 pg*hr/ml, for example about 8000 pg*hr/ml to about 14000 pg*hr/ml. In a related embodiment, the above PK parameters result after administration of a composition in an amount sufficient to provide the subject with about 0.5 to about 1.4 mg of butorphanol moiety.

In another embodiment, where the drug being delivered is butorphanol, upon intranasal administration of a butorphanol composition of the invention to a subject, the subject exhibits one or more of: a Tₘₐₓ butorphanol plasma concentration of at latest about 0.25 hr; a Cₘₐₓ butorphanol plasma concentration of at least about 1900 pg/ml, for example about 1900 pg/ml to about 5500 pg/ml; and/or an AUC butorphanol plasma concentration of at least about 9000 pg*hr/ml, for example about 9000 pg*hr/ml to about 13000 pg*hr/ml. In a related embodiment, the above PK parameters result after administration of a composition in an amount sufficient to provide the subject with about 0.5 to about 1.4 mg of butorphanol moiety.

In another embodiment, where the drug being delivered is butorphanol, upon intranasal administration of a butorphanol composition of the invention to a subject, the subject exhibits one or more of: a Tₘₐₓ butorphanol plasma concentration of at latest about 0.25 hr; a Cₘₐₓ butorphanol plasma concentration of at least about 2500 pg/ml, for example about 2500 pg/ml to about 5500 pg/ml; and/or an AUC butorphanol plasma concentration of at least about 9000 pg*hr/ml, for example about 9000 pg*hr/ml to about 13000 pg*hr/ml. In a related embodiment, the above PK parameters result after administration of a composition in an amount sufficient to provide the subject with about 0.5 to about 1.4 mg of butorphanol moiety.

In another embodiment, where the drug being delivered is butorphanol, upon intranasal administration of a butorphanol composition of the invention to a subject, the subject exhibits a plasma concentration of butorphanol of one or more of about 2800 to about 3300 ng/ml 30 minutes after administration, 1600 to about 2200 ng/ml 1 hour after administration, 1200 to about 1800 ng/ml 2 hours after administration, about 1400 to about 1600 ng/ml 4 hours after administration, and/or about 300 to about 800 ng/ml 6 hours after administration. In a related embodiment, the above PK parameters result after administration of a composition in an amount sufficient to provide the subject with about 0.5 to about 1.4 mg of butorphanol moiety.

In another embodiment, where the drug being delivered is butorphanol, upon intranasal administration of a butorphanol composition of the invention to a subject, the subject exhibits a plasma concentration of butorphanol of one or more of about 2900 to about 3200 ng/ml 30 minutes after administration, 1700 to about 2100 ng/ml 1 hour after administration, 1300 to about 1700 ng/ml 2 hours after administration, about 1450 to about 1550 ng/ml 4 hours after administration, and/or about 350 to about 750 ng/ml 6 hours after administration. In a related embodiment, the above PK parameters result after administration of a composition in an amount sufficient to provide the subject with about 0.5 to about 1.4 mg of butorphanol moiety.

In another embodiment, where the drug being delivered is hydromorphone, upon intranasal administration of a composition of the invention to a subject, the subject exhibits one or more of: a Tₘₐₓ hydromorphone plasma concentration of at latest about 1 hr; a Cₘₐₓ hydromorphone plasma concentration of at least about 2000 pg/ml, for example about 2000 pg/ml to about 10000 pg/ml; and/or an AUC hydromorphone plasma concentration of at least about 3000 pg*hr/ml, for example about 3000 pg*hr/ml to about 12000 pg*hr/ml. In a related embodiment, the above PK parameters result after administration of a composition in an amount sufficient to provide the subject with about 0.7 to about 1.8 mg of hydromorphone moiety.

In a related embodiment, where the drug being delivered is hydromorphone, upon intranasal administration of a hydromorphone composition of the invention to a subject, the subject exhibits one or more of: a Tₘₐₓ hydromorphone plasma concentration of at latest about 0.5 hr; a Cₘₐₓ hydromorphone plasma concentration of at least about 2500 pg/ml, for example about 2500 pg/ml to about 8000 pg/ml; and/or an AUC hydromorphone plasma concentration of at least about 3500 pg*hr/ml, for example about 3500 pg*hr/ml to about 11000 pg*hr/ml. In a related embodiment, the above PK parameters result after administration of a composition in an amount sufficient to provide the subject with about 0.7 to about 1.8 mg of hydromorphone moiety.

In another embodiment, where the drug being delivered is hydromorphone, upon intranasal administration of a hydromorphone composition of the invention to a subject, the subject exhibits one or more of: a Tₘₐₓ hydromorphone plasma concentration of at latest about 0.40 hr; a Cₘₐₓ hydromorphone plasma concentration of at least about 3000 pg/ml, for example about 3000 pg/ml to about 7000 pg/ml; and/or an AUC hydromorphone plasma concentration of at least about 4000 pg*hr/ml, for example about 4000 pg*hr/ml to about 10000 pg*hr/ml. In a related embodiment, the above PK parameters result after administration of a composition in an amount sufficient to provide the subject with about 0.7 to about 1.8 mg of hydromorphone moiety.

In another embodiment, where the drug being delivered is hydromorphone, upon intranasal administration of a hydromorphone composition of the invention to a subject, the subject exhibits one or more of: a Tₘₐₓ hydromorphone plasma concentration of at latest about 0.35 hr; a Cₘₐₓ hydromorphone plasma concentration of at least about 3200 pg/ml, for example about 3200 pg/ml to about 6000 pg/ml; and/or an AUC hydromorphone plasma concentration of at least about 5000 pg*hr/ml, for example about 5000 pg*hr/ml to about 9000 pg*hr/ml. In a related embodiment, the above PK parameters result after administration of a composition in an amount sufficient to provide the subject with about 0.7 to about 1.8 mg of hydromorphone moiety.

Delivery Device

Compositions of the present invention can be administered using any suitable intranasal delivery device. In one embodiment, the delivery device is a unit-dose delivery device. Delivery devices comprising any of the pharmaceutical compositions of various embodiments disclosed herein comprise embodiments of the invention. Non-limiting examples of suitable intranasal delivery devices, or components thereof, are disclosed in the following U.S. Patents and U.S. Patent Publications, each of which are hereby incorporated by reference herein in their entirety:

U.S. Pat. No. 4,946,069;

U.S. Pat. No. 5,307,953;

U.S. Pat. No. 5,368,201;

U.S. Pat. No. 5,395,032;

U.S. Pat. No. 5,427,280;

U.S. Pat. No. 5,482,193;

U.S. Pat. No. 5,584,417;

U.S. Pat. No. 5,813,570;

U.S. Pat. No. 5,893,484;

U.S. Pat. No. 5,944,222;

U.S. Pat. No. 5,964,417;

U.S. Pat. No. 5,967,369;

U.S. Pat. No. 6,062,433;

U.S. Pat. No. 6,257,454;

U.S. Pat. No. 6,626,379;

U.S. Pat. No. 6,321,942;

U.S. Pat. No. 6,367,473; and

U.S. 2003/0163099.

The delivery device can be filled with single or multidose amounts of opioids. In one embodiment, the vessel or container holding the pharmaceutical composition and its sealing means are sterilizable. In one such embodiment, the parts of the device that are in contact with the pharmaceutical composition can be constructed and assembled in a configuration so as to allow for sterilization. Devices with one or more unit-dose(s) can be sterilized either before or after filling and/or packaging, employing methods and technology that are well known in the art. Individual devices can be packaged, sterilized and shipped; alternatively, entire shipping and storage packages can be sterilized at once, and the devices removed individually for dispensing, without affecting the sterility of the remaining units.

In one embodiment, the volume of liquid administered to a subject to provide a therapeutically effective dose of a composition of the invention is about 0.025 ml to about 2 ml, about 0.25 ml to 1 ml, or about 0.05 ml to about 0.15 ml. However, the pharmaceutical compositions of the present invention are not limited to any one particular volume.

In another embodiment, a composition of the invention, upon being discharged from an intranasal spray device at a spray distance of 1 cm from a detection laser, for example at a discharge volume of about 100 μl per spray, exhibits a droplet size distribution having a mean Dv10 of about 10 to about 25 μm, about 11 to about 22 μm, or about 12 to about 20 μm; a mean Dv50 of about 15 to about 70 μm, about 17 to about 68 μm, or about 40 to about 50 μm; and/or a mean Dv90 of about 60 to about 130 μm, about 65 to about 120 μm, or about 80 to about 100 μm. In another embodiment, the spray has a mean span [(Dv90-Dv10/Dv50)] of about 1 to about 5, about 1.25 to about 4, or about 1.5 to about 3.

In a related embodiment, upon positioning the device 1 cm away from an impaction plate, actuating the device to produce a spray pattern onto the impaction plate, and measuring the diameter of the spray pattern the spray pattern has a maximum diameter (Dₘₐₓ) of about 1 to about 4 cm, about 2 to about 3 cm or about 2.2 to about 2.5 cm, for example about 2.3 cm. In another related embodiment, the spray has a minimum diameter (Dₘᵢₙ) of about 1 to about 3 cm, about 1.5 to about 2.8 cm or about 1.8 to about 2.3 cm, for example about 2.1 cm.

In another embodiment, a composition of the invention, upon being discharged from an intranasal spray device at a spray distance of 3 cm from a detection laser, exhibits a droplet size distribution having a mean Dv10 of about 9 to about 20 μm, about 9 to about 18 μm, or about 10 to about 15 μm; a mean Dv50 of about 20 to about 60 μm, about 25 to about 55 μm, or about 30 to about 40 μm; and/or a mean Dv90 of about 60 to about 130 μm, about 65 to about 120 μm, or about 80 to about 100 μm. In another embodiment, the spray has a mean span [(Dv90-Dv10/Dv50)] of about 1 to about 5, about 1.25 to about 4, or about 1.5 to about 3.

In related embodiment, upon positioning the device 3 cm away from an impaction plate, actuating the device to produce a spray pattern onto the impaction plate, and measuring the diameter of the spray pattern, the spray pattern has a maximum diameter (Dₘₐₓ) of about 4 to about 7 cm, about 4.5 to about 6 cm or about 4.8 to about 5.5 cm, for example about 5.2 cm. In another related embodiment, the spray has a minimum diameter (Dₘᵢₙ) of about 3 to about 6 cm, about 3.5 to about 5 cm or about 4.2 to about 4.8 cm, for example about 4.6 cm.

In another embodiment, a composition of the invention, upon being discharged from an intranasal spray device at a spray distance of 5 cm from a detection laser, exhibits a droplet size distribution having a mean Dv10 of about 9 to about 20 μm, about 9 to about 18 μm, or about 12 to about 17 μm; a mean Dv50 of about 20 to about 60 μm, about 25 to about 55 μm, or about 30 to about 40 μm; and/or a mean Dv90 of about 60 to about 90 μm, about 65 to about 85 μm, or about 67 to about 75 μm. In another embodiment, the spray has a mean span [(Dv90-Dv10/Dv50)] of about 1 to about 4, about 1.25 to about 3, or about 1.5 to about 2.

In related embodiment, upon positioning the device 5 cm away from an impaction plate, actuating the device to produce a spray pattern onto the impaction plate, and measuring the diameter of the spray pattern the spray pattern has a maximum diameter (Dₘₐₓ) of about 6 to about 9 cm, about 6.5 to about 8.5 cm or about 7 to about 8 cm, for example about 8 cm. In another related embodiment, the spray has a minimum diameter (Dₘᵢₙ) of about 6 to about 8 cm, about 6.5 to about 7.5 cm or about 6.6 to about 7.3 cm, for example about 7.2 cm.

Administration

Compositions of the present invention can be used to elicit analgesia or an analgesic response to relieve or alleviate pain in a subject, for example a mammal. Non-limiting diseases and/or conditions that cause pain include, cancer, arthritis, neurological diseases, heart attacks, trauma, childbirth, migraines, or surgery, dental procedures, etc.

Compositions of the present invention may also be used to produce anesthesia or an anesthetic response to permit the performance of surgery or other otherwise painful procedures. Mammals include, for example, humans, as well as pet animals such as dogs and cats, laboratory animals, such as rats and mice, and farm animals, such as horses and cows.

### EXAMPLES

The examples below are for illustrative purposes only and are not to be construed as limiting the invention in any manner.

### Example 1

The experiments described in this example compared bioavailability and other parameters of a butorphanol formulation when administered using a unit-dose or multi-dose delivery device. The butorphanol formulation used for this example (STADOL NS®) contained 10 mg butorphanol tartrate, 6.5 mg sodium chloride, 1.0 mg citric acid, 0.20 mg benzethonium chloride in purified water with 1.2 mg sodium hydroxide and hydrochloric acid added to adjust the pH to 5.0.

The multi-dose sprayer purported by its label to administer 0.1 ml of liquid composition by metering upon activation by the user. The unit-dose spray device was a disposable intranasal applicator that is commercially available from Pfeiffer of America under the designation “Unitdose Second Generation.” Each of the Pfeiffer spray applicators was charged with sufficient liquid to deliver a 0.1 ml dose of the butorphanol formulation. The associated glass containers were filled using a pipette under clean conditions, sealed and assembled to the applicator. Each of the applicators was weighed prior to use and after use. Qualified medical personnel administered one dose into each nostril, after which the applicator was recovered for weighing. In the case of the unit-dose applicators, two devices were used for each patient for a total of 2 mg butorphanol tartrate. Amount of composition dispensed from each of the delivery devices was measured and is shown in Table 1 below.

TABLE 1
Sample Characteristics of Dose Weight Delivery

| Delivery | | mean wt. | std. | std. | | |
|---|---|---|---|---|---|---|
| System | N | gms | dev. | error | minimum | maximum |
| Unit-Dose | 23 | 0.206 | 0.00660 | 0.00138 | 0.193 | 0.223 |
| Multi-Dose | 24 | 0.180 | 0.0285 | 0.00582 | 0.114 | 0.220 |

Unit-Dose:

The statistical comparison of dose 1 and dose 2 for the unit-dose delivery system was done using a paired t-test. Analysis of the data indicated that the difference between the mean sprays of the two applications using the Pfeiffer device was not statistically significant (t=1.0; p=0.3). The sample of 23 sprayers (actually 23 sets of 2 sprayers, since they were single-dose) had a mean total dose for two sprays of 0.206 grams with a standard deviation of 0.00660 grams.

Multiple-Dose:

The total dose dispensed by two sprays was recorded. The sample of 24 multi-dose sprayers had a mean total dose for two sprays of 0.180 grams with a standard deviation of 0.0285 grams.

Comparison of Average Total Dose:

The two-sample t-test for the comparison of the unit-dose and multi-dose sprayers indicated a statistically significant difference between the mean total doses taking into account the size of the sample. The unit-dose mean total dose was significantly closer to the prescribed target dose than the multi-dose mean total dose (t=4.3; p<0.001). A 95% confidence interval for the difference in means is (0.0140, 0.0380).

Comparison of Variability:

The F test for the comparison of variances revealed that the variability in the total doses dispensed by the multi-dose sprayer was significantly higher than the variability in weights dispensed by the unit-dose sprayer (F=18.7; p<0.001). The variability in the multi-dose sprayer is 18.6 times that of the unit-dose sprayer. High variability in dose delivery leads to higher rates of adverse drug effects at excessive dose and inadequate treatment if the dose is low. Both consequences harm the patient hence the goal is to precisely deliver the prescribed dose.

Comparison of Each Sprayer to the Standard of 0.2 Grams

A t-test was used in each case to compare the observed sample mean to the desired weight of 0.2 grams. The unit-dose sprayer dispensed a mean total weight that was significantly higher than the goal of 0.2 grams (t=4.4; p<0.001). A 95% confidence interval for the mean total weight dispensed by the unit-dose sprayer is (0.203, 0.209). The multi-dose sprayer dispensed a mean total weight that was significantly lower than the goal of 0.2 grams (t=3.4; p<0.003). A 95% confidence interval for the mean total weight dispensed by the multi-dose sprayer is (0.168, 0.192). Based on the above, the unit-dose delivery system exhibits a much higher degree of accuracy in intranasally administering the volume of liquid composition corresponding to 0.1 gm: +3% vs. −10%.

Pharmacokinetics and Bioequivalence

Pharmacokinetics and bioequivalence (intrasubject variabilitiy) of the above-described butorphanol formulation upon administration using either the multi-dose or unit dose delivery device was assessed. The study was initiated with 16 subjects, 15 of which completed the study to provide data for this analysis; one subject dropped out after the second period. The following analysis considers both raw and normalized data, with the latter standardized with respect to the dose dispensed.

Intranasal administration of 2 mg dose of butorphanol tartrate using the unit-dose device produced a Tₘₐₓ of about 0.234 hr (range of about 0.083 to about 0.333 hr); a Cₘₐₓ of about 5230 pg/ml (range of about 2393 to about 8478 pg/ml); and an AUC(₀₋ₜ) of about 10661 pg*hr/ml (range of about 5351 to about 17722 pg*hr/ml). Intranasal administration of 2 mg dose of butorphanol tartrate using the STADOL NS® multi-dose device produced a Tₘₐₓ of about 0.245 hr (range of about 0.167 to about 0.333 hr); a Cₘₐₓ of about 4027 pg/ml (range of about 184 to about 7312 pg/ml); and an AUC(₀₋ₜ) of about 9329 pg*hr/ml (range of about 903 to about 15932 pg*hr/ml).

For both the raw and normalized data, log transformations are applied to the pharmacokinetic endpoints Cₘₐₓ, AUC(last), and AUC(inf). A mixed effects model was considered for each parameter. Fixed effects for the factors sequence (4 levels), period 3 levels) and device (2 levels) were included in the model. Additionally, gender, as well as the interactions between gender and each of sequence, period and device was included as a factor in each model to determine whether separate analyses would be necessary for males and females. A total of seven models were considered: Tₘₐₓ, log of raw Cₘₐₓ values, log of normalized Cₘₐₓ values, log transformed values for raw and normalized AUC(last), and log values for raw and normalized AUC_{(inf)}. In all cases, the interaction between gender and formulation was not significant, indicating that separate models for males and females were not warranted. In addition, the lack of significance of the effects included in each model indicate that there was no evidence of unequal carryover between the two delivery devices.

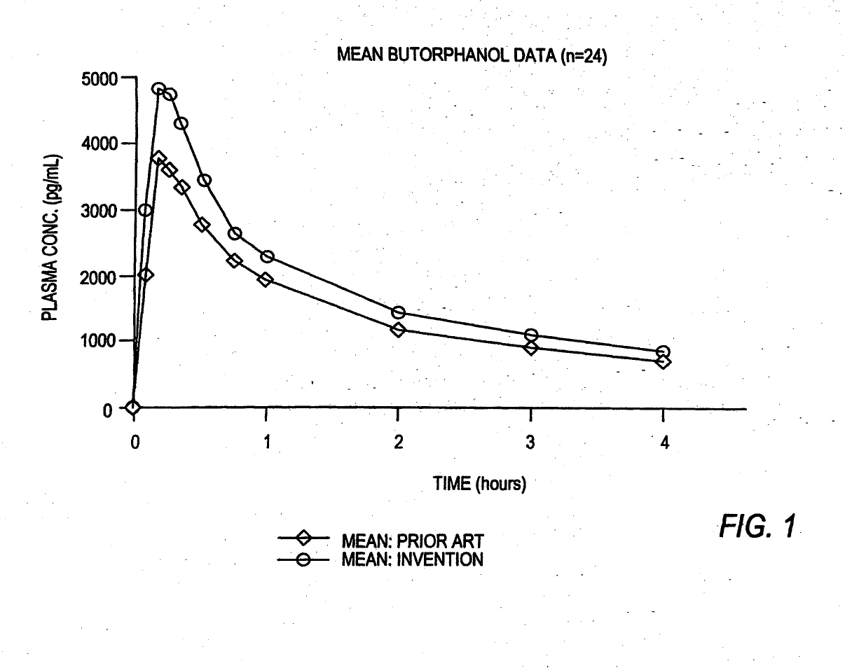

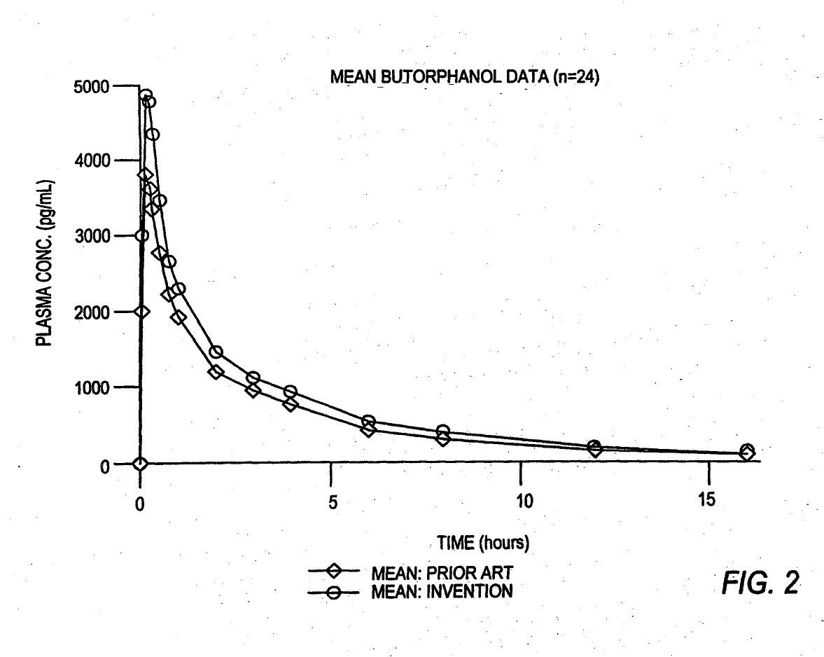

The mean levels of butorphanol from analysis of the subject's blood plasma reported in pg/ml is plotted against time in FIGS. 1 and 2. The concentration of drug after administration using the unit-dose device was unexpectedly higher than that after administration with the multi-dose device. The testing for bioequivalence was done using the method of two one-sided t-test (as described by Bolton, S., Pharmaceutical Statistics. Marcel Dekker, Inc., New York, 1997, pages 415 ff.). For each parameter, the 90% confidence interval for the ratio of PK parameters after administration of butorphanol using the unit-dose delivery device to the multi-dose delivery device appear in Table 2 below.

TABLE 2
Summary of the two one-sided hypothesis tests for PK parameters

| | Lower Conf Limit | Upper Conf Limit |
|---|---|---|
| | for Ratio of | for Ratio of |
| Parameter | Test/Reference | Test/Reference |
| Tmax | 0.749 | 1.132 |
| log (Cmax)* | 1.031 | 1.855 |
| log (AUClast)* | 1.037 | 1.540 |
| log (AUCinf)* | 1.050 | 1.461 |
| log (normCmax)* | 0.897 | 1.589 |
| log (AUClast)* | 0.921 | 1.290 |
| log (normAUCinf)* | 0.937 | 1.220 |

*Note: The actual confidence limits obtained for these parameters have been exponentiated since the data were log-transformed originally.

Since none of these confidence intervals for the non-standardized data are contained in the interval from 0.8 to 1.25, the conclusion is that the two delivery devices are not equivalent when compared on raw values. For Tₘₐₓ, the one-sided t-test for Hₒ: Test/Reference <0.8 is not rejected. Also, the tests of Hₒ: Test/Reference >1.25 are not rejected for any of the log-transformed raw values. While the normalization by dispensed doses does improve the comparability of the two delivery devices, two of the three parameters fail to reject the null hypothesis Hₒ: Test/Reference >1.25. Bioequivalence is supported only by the pair of one-sided tests for the normalized, log-transformed AUC(inf). Both one-sided t-test for each of the seven parameters have been performed at an alpha level of 0.05.

The data show that the FDA-approved STADOL NS® product that has been sold and dispensed for a number of years unexpectedly delivers below label strength. The degree of variability is also significantly greater than when the composition is administered using the unit-dose delivery device.

Equality of Variances

The Pitman-Morgan adjusted F test was used to compare variances of the unit-dose and multi-dose parameters. (See Chow, S-C. and Liu, J-P, Design and Analysis of Bioavailability and Bioequivalence Studies. Marcel Dekker, Inc., New York (2000)). Since this test could not be generalized to the three period design, the first two periods of the butorphanol trial were used, and for the purposes of this analysis, there are two devices, two periods, and two sequences. The Pitman-Morgan adjusted F test can be used even if the period effect is significant, and has a simplified form in the absence of period effects. Of the seven PK parameters considered, only Tₘₐₓ exhibited a significant period effect. Table 3 summarizes the results of the tests of equality. The null hypothesis is that the variances are equal, and small p-values are indicative of a departure from equality.

TABLE 3
Summary of the Pitman-Morgan's adjusted F tests for PK
parameters

| | Pitman-Morgan F | |
|---|---|---|
| Parameter | Value | p-value |
| Tmax | 0.3 | 0.6 |
| log (Cmax) | 11.3 | 0.005 |
| log (AUClast) | 30.1 | <0.0001 |
| log (AUCinf) | 15.3 | 0.002 |
| log (normCmax) | 8.4 | 0.01 |
| log (AUClast) | 23.7 | 0.0002 |
| log (normAUCinf) | 10.7 | 0.0005 |

The tests of equality variances indicate that for all PK parameters except Tₘₐₓ, the variabilities of the two devices are significantly different, with the unit dose system demonstrating much lower variability of drug levels in the plasma. While the normalization of the Cₘₐₓ, AUC₍ₗₐₛₜ₎ and AUC_{(inf)} parameters somewhat decreased the difference between the variances (as evidenced by slightly smaller F values), the variances were nonetheless significantly different. The variability associated with the unit-dose system was smaller than that of the multi-dose system of the prior art, which is consistent with the findings of the delivery volume weight study.

From the above, it is apparent that the dose weight/volume data is confirmed by the plasma level (PK) analysis. The multi-dose delivery device results in an area under the curve that is 90% of that of the unit-dose delivery device. Thus, the unit-dose delivery device achieves more than 10% higher area under the curve and more than 10% higher plasma levels as compared to the multi-dose delivery device. This difference is highly significant from a patient therapy standpoint. When FDA-prescribed bioequivalence statistical methods are applied, it is concluded that the products as administered to patients are not equivalent.

### Example 2

Pharmacokinetic parameters of single doses of intranasal butorphanol tartrate using a single-dose, metered sprayer were evaluated in a 24 subject, three-way crossover study. The intranasal formulation contained aqueous buffered solution (pH 5) having 0.2% sodium citrate and 0.2% citric acid and 1 mg of butorphanol tartrate per ml. The composition did not have a preservative. Each volunteer received three treatments: (1) 2 mg of i.v. butorphanol, (2) 2 mg of intranasal butorphanol, and (3) 1 mg of intranasal butorphanol. Each treatment was separated by a 6 day washout period. Venous blood samples were collected predose and at 5, 10, 15, 20, 30 and 45 minutes and 1, 2, 3, 4, 6, 8, 12 and 16 hours post dose. Pharmacokinetic parameters are shown in Table 4.

TABLE 4
Pharmacokinetic parameters

| Parameter | 2 mg i.v. | 1 mg intranasal | 2 mg intranasal |
|---|---|---|---|
| Tₘₐₓ | 0.285 ± 0.06 | 0.436 ± 0.24 | 0.381 ± 0.23 |
| Cₘₐₓ (ng/ml) | 10.32 ± 2.7 | 1.67 ± 0.7 | 3.38 ± 1.3 |
| AUC₀₋ₜ (ng * hr/ml) | 12.59 ± 2.3 | 4.88 ± 1.5 | 9.51 ± 1.9 |
| F (%) | Assume 100% | 80.2 ± 29.1 | 77.6 ± 18.3 |

Example 3

Pharmacokinetic parameters of single doses or multi doses of intranasal butorphanol tartrate using a single-dose, metered sprayer were evaluated in a 12 subject, two-way crossover study. The butorphanol formulation used was as described in Example 2. Each volunteer received either 1 or 2 mg of intranasal butorphanol as a single does (Treatment A) and 1 or 2 mg of intranasal butorphanol every six hours for seven doses (Treatment B). The butorphaonol composition was substantially as described in Example 2. During phase 1, 12 subjects received a single 1 mg dose. During phase 2, those who received the 1 mg single dose received 1 mg every six hours for seven doses. During phase 3, those who received the 2 mg single dose received 2 mg every six hours for seven doses. Serial blood samples were collected over 12 hours. Pharmacokinetic parameters are show in Table 5.

TABLE 5
Pharmacokinetic Parameters

| | | | | Multiple |
|---|---|---|---|---|
| | Single Dose | Multiple Dose | Single Dose | Dose |
| Parameter | 1 mg | 1 mg q 6 hr | 2 mg | 2 mg q 6 hr |
| Tₘₐₓ | 0.33 | 0.5 | 0.25 | 0.5 |
| | (0.17-1.02) | (0.25-3.0) | (0.167-0.5) | (0.25-0.75) |
| Cₘₐₓ | 1.46 | 1.55 | 4.13 | 3.80 |
| (ng/ml) | | | | |
| AUC₀₋ₜ | 3.80 | 7.21 | 9.55 | 15.75 |
| (ng * hr/ml) | | | | |

### Example 4

A test was performed to assess spray pattern characteristics and particle size distribution of spray of one composition of the invention. An intranasally deliverable composition was prepared comprising 10 mg butorphanol tartrate, 6.5 mg sodium chloride, 1.0 mg citric acid, and approximately 1.2 mg sodium hydroxide and hydrochloric acid or sodium hydroxide added to adjust the pH to 4.8-5.2 and QS to 1.0 mL with WFI.

Aliquots of the composition were loaded into the Pfeiffer Unitdose Second Generation device (100 μl per dose). Spray patterns were characterized at a spray distance of 1, 3 and 5 cm from an impaction plate, measured from the tip of the spray nozzle to the impaction plate. Data for longest (Dₘₐₓ) and shortest (Dₘᵢₙ) diameters and ovality (D_{max/Dmin}) for each spray were determined. Results are shown in Table 6 below.

TABLE 6
Spray Pattern Results

| Spray | Mean Dₘₐₓ (cm) | Mean Dₘᵢₙ (cm) | Mean Ovality |
|---|---|---|---|
| Distance | (range) | (range) | (range) |
| 1 cm | 2.3 (2.2-2.4) | 2.1 (2.0-2.2) | 1.1 (1.0-1.2) |
| 3 cm | 5.2 (4.2-6.1) | 4.6 (3.8-5.8) | 1.1 (1.0-1.3) |
| 5 cm | 7.9 (7.0-8.4) | 7.2 (5.8-8.0) | 1.1 (1.0-1.2) |

### Example 5

Droplet size distribution, after the composition was sprayed from a Pfeiffer Second Generation Unitdose spray device (100 μl per spray), was determined for the composition described in Example 4. Droplet size distribution was determined using a Malvern Spraytec with RT sizer software. Samples were measured at three distances of 1 cm, 3 cm, and 5 cm between the nozzle tip of the device and the detection laser beam which ran perpendicular to the direction of spray.

At a spray distance of 1 cm, the spray had a droplet size distribution having a mean Dv10 of about 15.45 μm (range of 13.70 to 19.98), a mean Dv50 of about 41.46 (range of 35.74 to 55.67) and a mean Dv90 of about 93.88 μm (range of 69.55 to 117.15). The spray had a mean span [(Dv90-Dv10/Dv50)] of about 1.76 (range of 1.55-1.91).

At a spray distance of 3 cm, the spray had a droplet size distribution having a mean Dv10of about 13.83 μm (range of 11.84 to 15.68), a mean Dv50 of about 35.29 μm (range of 29.46 to 41.69) and a mean Dv90 of about 90.80 μm (range of 71.2 to 122.42). The spray has a mean span [(Dv90-Dv10/Dv50)] of about 2.17 (range of 1.92-2.56).

At a spray distance of 5 cm, the spray had a droplet size distribution having a mean Dv10 of about 15.82 μm (range of 14.38 to 17.17), a mean Dv50 of about 32.96 μm (range of 31.03 to 35.32) and a mean Dv90 of 71.85 μm (range of 61.64 to 83.68). The spray had a mean span [(Dv90-Dv10/Dv50)] of about 1.69 (range of 1.50-1.90).

### Example 6

Hydromorphone Intranasal Solution

Hydromorphone HCl was formulated in a liquid composition. Each 1 ml of nasal spray solution contained 10 mg hydromorphone HCl with 0.2% sodium chloride, 0.2% sodium citrate, 0.2% citric acid solution, and sterile water (i.e., water for injection, USP), accepted antioxidant concentration and buffer in pharmaceutical products. The pH of this formulation was approximately pH 4.0. This formulation was used in the hydromorphone clinical study below.

A protocol was designed to determine the bioavailability of hydromorphone HCl by the IM and IN routes by comparing the pharmacokinetics of intramuscularly administered hydromorphone HCl and intranasally administered hydromorphone HCl to hydromorphone HCl administered via the IV route. Specifically, the objectives of this study were: (1) to compare the pharmacokinetics of hydromorphone via intranasal, intramuscular, and intravenous administration of a 2 mg dose of hydromorphone HCl, and (2) to evaluate the bioavailability of 2 mg hydromorphone HCl after intranasal, IM and IV routes of administration using a standard three-period, crossover design.

The above composition was used to fill the required number of unit-dose, metered sprayers commercially produced and sold by Pfeiffer of America, Inc.

Nine healthy male subjects between the ages of 22 and 33 years participated in this inpatient study. Study participants were selected based on inclusion/exclusion criteria, history and physical exam, laboratory tests, and other customary procedures. Subject demographics were recorded. These included age range: 22-33 years; height range: 168-188 cm; weight range: 70.3-95.3/kg; origin: six Caucasian, two Asian, one Native American; all were non-smokers. All nine of the subjects completed the study according to the protocol. Each of the subjects received 3 doses of 2 mg of hydromorphone HCl on three separate occasions. No clinically significant protocol violations occurred during this study. Because the inclusion criteria mentioned abstinence from prescription and non-prescription drugs prior to and during the study, any medications taken in the 14 days before the study and during the study were noted.

Clinical Trials

Study Drug Formulation: hydromorphone HCl for intranasal administration was supplied by the University of Kentucky College of Pharmacy. hydromorphone HCl for intravenous administration was supplied as Dilaudid® 1 mg/mL for subjects 1, 3, 8, and 9 on the first day and for subjects 2, 4, 5, 6, 7 on the second study day. Hydromorphone HCl for intramuscular administration was supplied as Dilaudid® 4 mg/mL for subjects 2, 4, 5, 6 and 7 on the first study day and for subjects 1, 3, 8 and 9 on the second study day. Free base content was 1.77 mg or 88.7% of stated hydromorphone HCl strength (from molecular weights: 321.8−36.46=285.34; 285.34/321.8=88.7%). To summarize, the dosages for each of the three routes of administration were as follows.

Treatment A: 2 mg intravenous hydromorphone HCl

Treatment B: 2 mg intramuscular hydromorphone HCl

Treatment C: 2 mg intranasal hydromorphone HCl

Study Drug Administration

On days 1 and 8, 2 mg of hydromorphone HCl was given intravenously or intramuscularly in random order following an overnight fast. On day 15, 2 mg of hydromorphone HCl was given intranasally following an overnight fast (except for water ad lib). Subjects were not permitted to recline for 4 hours following drug administration and remained fasting for 4 hours (until lunch) on these study days.

Meals and snacks prepared by the University of Kentucky Hospital Dietetics and Nutrition department were provided for each subject. Subjects were instructed to eat all of their meals. All subjects received identical meals and snacks on each of the treatment days, but received different meals on non-treatment study days.

Weight, blood pressure, and pulse were measured prior to dosing and at the end of the study. Blood pressure and pulse rate were measured with the subjects seated in an upright position before any corresponding blood sample was collected. Blood pressure and pulse rate were measured and recorded on the same arm throughout the study at 0 (pre-dose) and 30 minutes, 1, 2, 4, 8 and 16 hours. Spontaneously reported adverse events were recorded by the subjects throughout the study; adverse events were also elicited by non-directed interviews.

Blood samples were collected during the study from each subject according to the following schedule: 0 (pre-dose), 5, 10, 15, 20, 30 and 45 minutes, and 1, 2, 3, 4, 6, 8, 12 and 16 hours following hydromorphone HCl administration. The beginning of the IV administration was considered time zero. After collection, the blood was centrifuged in a refrigerated centrifuge at 4° C. to separate the plasma and the cells and the plasma was transferred to polypropylene tubes. The plasma was stored at approximately −70° C. at the study site until shipped to an independent analytical service. The plasma was maintained frozen during shipping and upon arrival at the remote analytical facility, the samples were stored at approximately −20° C. until analyzed.

Bioanalytical Methods

LC/MS/MS Assay for Hydromorphone

Sample analysis was performed by an independent service in accordance with established protocols. Concentrations less than 20 pg/mL were reported as below quantitation limit (BQL). Samples with concentrations greater than 2,000 pg/mL were reanalyzed using a dilution so that the assayed concentration was within the range of 20 to 2,000 pg/mL. QC samples were also diluted. During the validation, the precision was expressed as the percent coefficient of variation (% CV) and the accuracy as the percent difference from the theoretical (same as relative error).

Pharmacokinetic Methods

Plasma concentration versus time data for hydromorphone were analyzed using non-compartmental pharmacokinetic methods. Maximum plasma concentration (Cₘₐₓ) and the corresponding sampling time (Tₘₐₓ ) were identified by observation. Concentration versus time data were plotted on a semi-logarithmic scale and the terminal log-linear phase was identified by visual inspection. The elimination rate constant (λ_{z}) was determined as the slope of the linear regression for the terminal log-linear portion of the concentration versus time curve. The terminal half-life value (t_{1/2}) was calculated as 0.693 divided by λ_{z}.

The area under the curve plotting plasma concentration versus time (AUC) was calculated by the trapezoidal rule and extrapolated to infinite time. The AUC to the last time point (AUC₀₋ₗₐₛₜ) was computed by the trapezoidal rule. Mean plasma concentrations were calculated for graphical presentation only. Data included in the mean calculation were for samples with measurable concentrations drawn within 5% of the nominal sampling time.

Safety Results

Results of the clinical measurement of vital signs and body weight exams were recorded and nasal exams were performed. A review of this data failed to reveal any clinically significant safety concerns. There were no serious adverse events and no subjects were discontinued due to adverse effects.

Bioanalytical Results

Hydromorphone in Plasma by LC/MS/MS

Results from the control samples and calibration curves analyzed with the study samples and the method validation was reported. The overall CV, which reflects precision, was <7.4% for the QC samples. The percent recovery ranged from 94.5 to 100 1% for QC concentrations 200.0, 500.0, and 1000 which reflects accuracy was <6% for the QC samples.

Pharmacokinetic Results

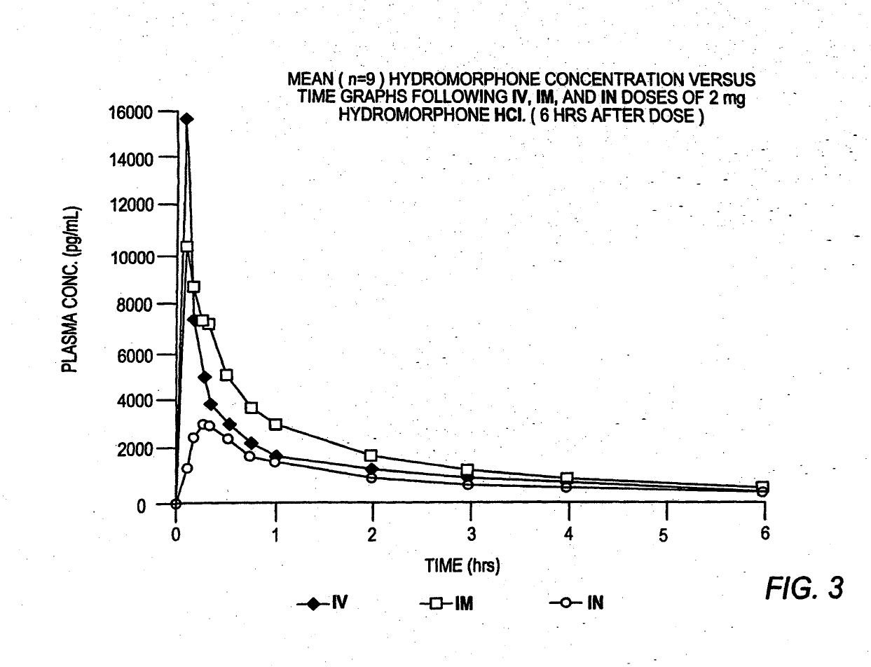

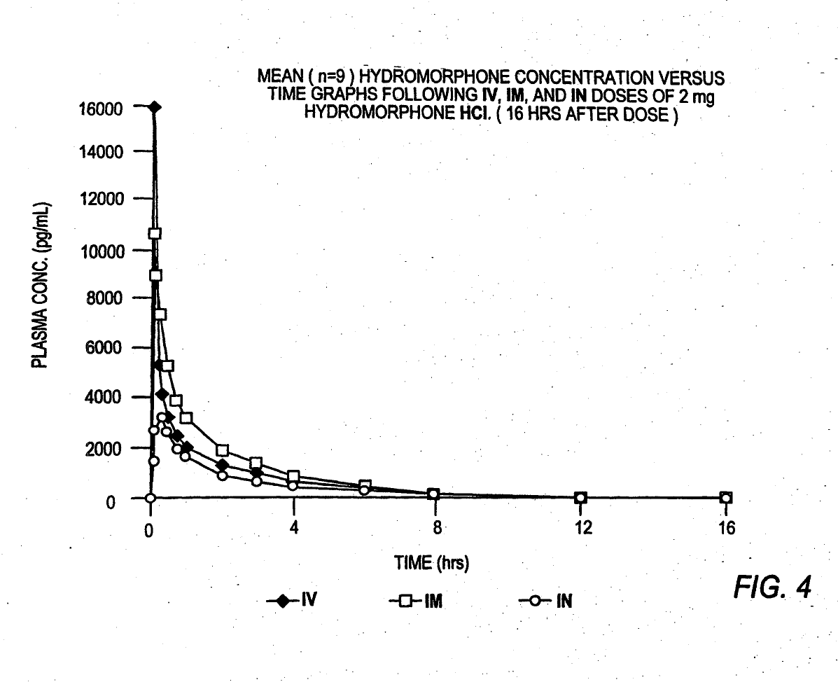

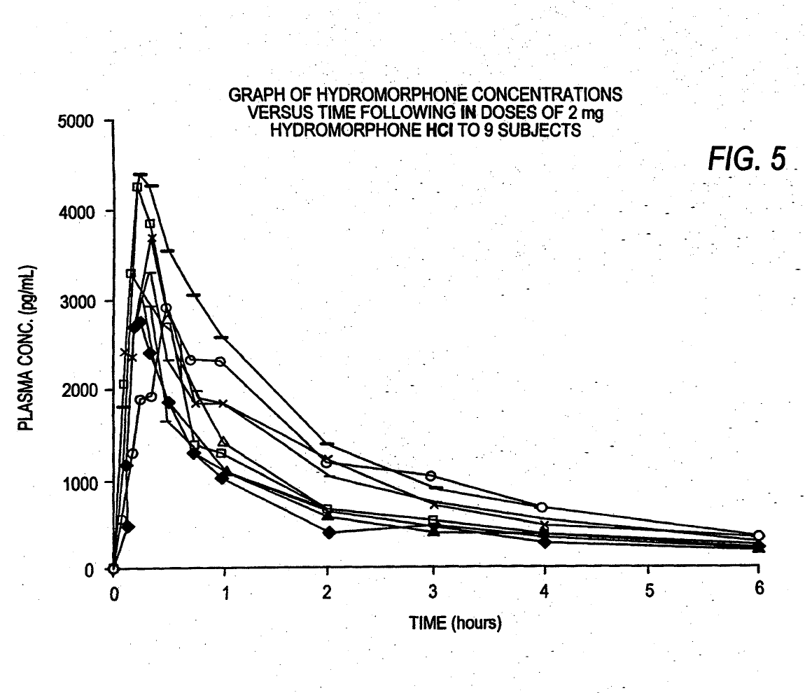

The plasma hydromorphone concentrations and actual collection times for each of the 9 subjects was tabulated and plasma concentration-time curves for each of the 9 subjects were prepared. Mean concentration-time curves of FIGS. 3 and 4 are representative for most subjects (mean data tabulation). FIG. 3 is a plot of the mean (n=9) hydromorphone concentration versus time graphs following IV, IM and IN doses of 2 mg hydromorphone HCl during the 6 hours after dose; FIG. 4 is the same data plotted for 16 hours after the dose. Curves for all subjects for 6 hours after the IN dose appear in FIG. 5 as a graph of hydromorphone concentrations versus time following IN doses of 2 mg hydromorphone HCl to 9 subjects.

Non-compartmental pharmacokinetic analysis was used to evaluate the plasma concentration versus time curves of hydromorphone following single 2 mg doses of hydromorphone HCl by intravenous (IV), intramuscular (IM), and intranasal (IN) routes. Individual plasma hydromorphone concentrations versus time profiles for all subjects were recorded; and a complete listing of individual and mean pharmacokinetic parameters for all 9 subjects was calculated.

Data are shown in Table 7, below. Rapid absorption of hydromorphone was observed after the IM and IN doses. The Tₘₐₓ values were approximately 9 and 18 minutes, on average, for the IM and IN doses, respectively. The hydromorphone Cₘₐₓ and AUCs were significantly higher after IM and IV administration compared to IN administration. Mean plasma half-lives and clearance (after correcting for bioavailability) were similar for all three treatments.

The arithmetic mean value of absolute bioavailability of hydromorphone from the IN formulation is 64%. The range was 50% to 81% bioavailability compared to the IV dose. The apparent bioavailability of the IM hydromorphone was about 30% greater than that of the same dose of IV administration. The source of this aberrant phenomenon was not found, but unusual distribution phenomena after parenteral administration have been reported by others working in this field. Pharmacokinetic data are shown in Table 7.

TABLE 7
Pharmacokinetic Data

| Parameter | 2 mg i.v. | 2 mg i.m. | 2 mg i.n. |
|---|---|---|---|
| Tₘₐₓ (hr) | 0.094 | 0.148 | 0.305 |
| | (0.083-0.167 | (0.083-0.333) | (0.25-0.333) |
| Cₘₐₓ (pg/ml) | 15547 | 10777 | 3438 |
| | (6949-28943) | (6752-18119) | (2760-4417) |
| AUC₍₀₋ₜ₎ | 9899 | 12984 | 6188 |
| (pg * hr/ml) | (8553-11540) | (10583-15310) | (4250-8732) |
| AUC_{(0-∞)} | 10393 | 13471 | 6681 |
| (pg * hr/ml) | (8788-12920) | (11167-16721) | (4359-9098) |

Statistical Evaluation

Various pharmacokinetic parameters were analyzed to evaluate the effect of routes of administration and to test for period and sequence effects. The analysis of this pilot data is considered in two parts: the first part considers only the first two periods and includes the factors of treatment, sequence (i.e., a test of carryover effects) and period; the second part contains all three periods and treatments, but ignores the effects of sequence and period. The 2-period analysis is noted in Table 8 as period 1 vs. 2 and the last column contains the 3-period model.

There are even more significant treatment effects for these nine outcomes. Post-hoc analyses are based on Fisher's least significant difference procedure and displayed in Table 8. In light of the fact that there were no significant period or sequence effects (using an alpha level of 0.05), and since this is a pilot project, it is arguable that the above analysis is appropriate.

Since the Cₘₐₓ value for Subject 07 was beyond 2 standard deviations of the mean with all measurements included, there is an objective method for omitting this value for this subject. Analyses with and without this outlier gave the same result.

TABLE 8
Summary of significance levels from 2-period and 3-period model

| | Sequence | Period | Treatment | Treatment |
|---|---|---|---|---|
| Parameter | (1 vs 2) | (1 vs 2) | IV vs IM | (IV vs IM vs IN) |
| Tₘₐₓ | NS* | NS | NS | .0001 |
| Cₘₐₓ | NS | 0.32 | 0.71 | .0001 |
| Cₘₐₓ(omit | NS | 0.62 | NS | .0001 |
| outlier) | | | | |
| AUC₀₋ₜ | NS | NS | .0001 | .0001 |
| AUC_{0-∞} | NS | NS | .0001 | .0001 |
| t_{1/2} | NS | NS | NS | NS |
| CL/F | NS | NS | .0001 | .0001 |
| Dose | NS | NS | .0001 | .0001 |
| λ_{z} | NS | NS | NS | NS |

*All p-values reported as NS are >0.1.

In this study of nine healthy male subjects that received 2 mg hydromorphone HCl by IV, IM and IN routes, comparisons between the IM and IN doses for purposes of bioequivalence could not be completed when it was found that the hydromorphone concentrations for the IM dose were markedly different as compared to those from the IN doses.

Noncompartmental analysis of the pharmacokinetic data gave results similar to previous studies with respect to half-lives, clearance, rapid distribution into the tissues, and large apparent distribution volume (Parab et al. 1988; till et al. 1991), although comparisons between this study and previous studies should be done with caution because of differences in analytical techniques. Hydromorphone is well absorbed by the nasal route. Intranasal bioavailability was approximately 64%, on average. Interindividual variation was smaller for Cₘₐₓ and Tₘₐₓ for the IN route compared to the IV and IM routes. Three compartment characteristics were suggested by the tri-phasic concentration versus time curves, but compartmental analysis was not performed.

After the short IV infusion, the hydromorphone concentrations peaked at the end of the infusion as expected in all but one subject. Peak concentrations after the IM dose were unexpectedly rapid and precluded the analysis of the data for showing the bioequivalence of the IM and IN doses, and that analysis was not pursued.

Pharmacokinetic parameter estimates yielded CVs less than 27% for IN parameters except for Vₛₛ (CV 46%). Estimates of within-subject variability were smaller than estimates for published studies of IV hydromorphone (Parab et al.; Hill et al.; Vallner et al.). Using a crossover design and standardizing meal times in this study likely helped to lower within-subject variability.

Variabilities in CL and Vₛₛ estimates are less after the IV dose compared to the IN dose. The reduced variability is expected since IV dosing avoids between-subject variability in absorption and first-pass metabolism.

Adverse events were less frequent and milder after the IN dose compared to the IV and IM doses. Assuming a dose-response relationship, this effect is believed to be attributable to the fact that the bioavailability of the IN dose was less and the peak concentration lower, so the subjects effectively received a lower dose that was more slowly absorbed. Nasal irritation was not observed with the exception of a bad taste in the throat reported by most subjects after the IN dose. In summary, hydromorphone is well absorbed by the nasal route with bioavailability of 64%. Cₘₐₓ and Tₘₐₓ were similar for IM and IV routes.

Hydromorphone HCl produced no systemic adverse events beyond those commonly experienced by injection. After single IN doses the subjects complained of bitter taste as the only local administration effect of the formulation. The bitter taste can be masked by the addition of a sweetener to the formulation. Detailed nasal examination demonstrated no pathology of the naso-pharynx after single administration of the hydromorphone HCl formulations.

What is claimed is:

1.-26. (canceled)

27. A method for providing analgesia, the method comprising intranasally administering to subject in need of analgesia, using an intranasal unit dose delivery device, a pharmaceutical composition comprising
an effective amount of hydromorphone or a pharmaceutically acceptable salt thereof in a sterilized, buffered, preservative-free liquid nasal carrier, wherein the composition, when administered to the subject provides:
a Tₘₐₓ hydromorphone plasma concentration of at most about 1 hr;
a Cₘₐₓ hydromorphone plasma concentration of about 2000 pg/mL to 10,000 pg/mL; and
an AUC₍₀₋ₜ₎ hydromorphone plasma concentration of about 3000 pg*hr/mL to 12,000 pg*hr/mL.

28. The method of claim 27, wherein the unit dose delivery device comprises one or more sealed vessels containing the pharmaceutical composition, such that when the pharmaceutical composition is discharged from the unit dose delivery device at a spray distance of 3 cm from a detection laser to produce a spray plume, the spray plume has a mean Dv10 of about 9 to 20 μm, a mean Dv50 of about 20 to 55 μm, a mean Dv90 of about 60 to 100 μm, and a mean span [(Dv90-Dv10/Dv50)] of about 1 to 5.

29. The method of claim 28, wherein the device, when discharged at a spray distance of 3 cm from an impaction plate, the resulting spray pattern has a maximum diameter (Dₘₐₓ) of about 4 to 7 cm; and a minimum diameter (Dₘᵢₙ) of about 3 to 6 cm.

30. The method of claim 27, wherein the hydromorphone or the pharmaceutically acceptable salt thereof is present in the pharmaceutical composition in a concentration of from about 1 to 20 mg/mL.

31. The method of claim 27, wherein the liquid nasal carrier comprises citric acid and water, and the composition has a pH of about 3 to 7.

32. The method of claim 27, wherein the pharmaceutical composition is a sterile solution or suspension.

33. The method of claim 27, wherein the amount of hydromorphone or the pharmaceutically acceptable salt thereof delivered by the intranasal unit dose delivery device is from about 1 to 15 mg.

34. The method of claim 27, wherein, upon intranasal administration of the composition to the subject, the subject exhibits a Tₘₐₓ hydromorphone plasma concentration of at most about 0.4 hour.

35. An intranasal unit-dose delivery device comprising one or more sealed vessels containing a sterilized, preservative-free pharmaceutical composition comprising an effective amount of hydromorphone and liquid nasal carrier, wherein the composition,when discharged from the device at a spray distance of 3 cm from a detection laser to produce a spay plume, the spray plume has a mean Dv10 of about 9 to 20 μm, a mean Dv50 of about 20 to 55 μm, a mean Dv90 of about 60 to 100 μm, and a mean span [(Dv90-Dv10/Dv50)] of about 1 to 5.

36. The device of claim 35, wherein each vial contains about 0.1 to 10 mg of hydromorphone.
